(19) United States
(12) Patent Application Publication
(10) Pub. No.: US 2022/0409543 A1
(43) Pub. Date: Dec. 29, 2022

(54) DEPOSITION OF NANOSUSPENSIONS OF ACTIVE PHARMACEUTICAL INGREDIENTS ON CARRIERS

(71) Applicant: MERCK PATENT GMBH, DARMSTADT (DE)

(72) Inventors: Dieter LUBDA, Darmstadt (DE); Gudrun BIRK, Darmstadt (DE)

(73) Assignee: MERCK PATENT GMBH, DARMSTADT (DE)

(21) Appl. No.: 17/776,368

(22) PCT Filed: Nov. 27, 2020
(86) PCT No.: PCT/EP2020/083573

§ 371 Date: May 12, 2022

(30) Foreign Application Priority Data
Dec. 2, 2019 (EP) 19212873.4

(51) Int. Cl. A61K 9/16; A61K 31/496

(52) U.S. Cl. CPC A61K 9/1694; A61K 9/1652; A61K 31/496

(57) ABSTRACT

The present invention provides a method for preparing a pharmaceutical composition of a pharmaceutical ingredient (API) which is loaded on a carrier and stabilized therethrough. In particular, the present invention relates to a composition of a poorly soluble nanoparticulated API on a carrier in the dry state and which is processed as pharmaceutical formulation of said API with improved release profile and bioavailability.

The present invention provides a method for preparing a pharmaceutical composition of a poorly soluble pharmaceutical ingredient (API) which is loaded on a carrier and stabilized therethrough. In particular, the present invention relates to a composition of a nanoparticulated API on a carrier in the dry state and which is processed as pharmaceutical formulation of said API with improved release profile and bioavailability.

### BACKGROUND OF THE INVENTION PROBLEM TO BE SOLVED

Poor drug solubility combined with low bioavailability remains a significant and common problem for pharmaceutical industry. In principle, for the bioavailability of an active pharmaceutical ingredient (API) solubility and dissolution rate are basic parameters. If the bioavailability of poorly soluble drugs shall be improved, these two factors have to be influenced. Therefore, many different drugs have been studied in the past in this context.

Another factor influencing the bioavailability consists in the uptake of the active substance into the metabolism and thus in the transition of the active ingredient into the body fluids by which the active ingredient can reach the site of action. Depending on the physical and chemical properties of the active ingredient, it must be provided in a specially adapted formulation.

Knowing the physicochemical characteristics of the active ingredient (solubility, permeability, particle size distribution, polymorphism, etc.) is a prerequisite to this process, and especially helps in placing the molecule in the biopharmaceutical classification systems (BCS). In this context, the ability of lipid-based formulations to facilitate gastrointestinal absorption of many poorly soluble drug candidates belonging to the BCS classes II and IV have been thoroughly documented in the published literature. However, a considerable gap still exists between the knowledge of this technology and the know-how required for its application. [Hauss, David; “Oral lipid-based formulations”; Advanced Drug Delivery Reviews 59 (2007) 667-676]. In the meantime, since the publishing of this review, the situation has not changed significantly, although 40% of all marketed drugs are among those of classes BCS II and IV drugs, and this is an increasing problem because up to 80% of all pharmaceutical development substances belong to these BCS classes.

Furthermore, the pharmaceutical industry is facing many challenges:
the need to develop innovative products much faster,
the need to differentiate own products from those of the competitors,
increasing R&D costs,
high failure rates during development due to substance structures generating “poor solubility or/and low bioavailability”.

Already more than 100 years ago, Noyes and Whitney [A. A. Noyes, W. R. Whitney; “The rate of solution of solid substances in their own solutions”; J. Am. Chem. Soc., 19 (1897), pp. 930-934] investigated the relationship between dissolution and solubility of solids in solvents and presented the relationship in a mathematical equation. Later the relationship between dissolution rate and solubility was characterized by the modified Noyes-Whitney equation [Nernst, W., 1904. “Theorie der Reaktionsgeschwindigkeit in heterogenen Systemen”; Z. Phys. Chem. 47, 52-55]:

$$\frac{dQ}{dt} = \frac{D * A ( {C}_{s} - {C}_{b} )}{h}$$

dQ/dt=dissolution rate

D=diffusion coefficient

h=diffusion layer thickness

Cₛ=solubility

C_{b}=bulk solution concentration

A=surface area of particle

Already in this equation, in addition to the diffusion coefficient, the surface of the active substance to be dissolved as well as the solubility are the most important variables, by which the dissolution rate can be influenced.

Recently, a variety of technologies have been developed and efforts have been made to improve the bioavailability of poorly soluble BCS Class II and IV drugs, but these approaches, despite many advantages, have numerous disadvantages. In particular, it is desirable to achieve an improvement in the bioavailability as possible by adding only few additives, but preferably without the addition of additives.

Now, returning back to the modified Noyes-Whitney equation, one way

for improving the solubility of a poorly soluble drug should be to increase the surface area (A) of the poorly soluble solid active ingredient. This could be done by micronization or nanonization methods. One straight forward possibility to reduce particle size and increase the surface area is to use a milling process. Another known technology for this is for example co-grinding in the presence of supercritical fluids.

Another option to improve the solubility (Cs) of these poorly soluble actives is to change the properties of the liquid environment in which the actives shall be solved. This can be done by mixing the solvent with solubility enhancers, like cyclodextrins, or with lipidic formulations consisting for example of oils, surfactants, co-surfactants, co-solvents and solubilized drug substances, which are forming a self-emulsifying drug delivery system (SEDDS) or by a self-microemulsifying drug delivery system (SMEDDS), which is a modified SEDDS which can form fine oil-in-water droplets with a diameter size of less than 50 nm under mild agitation of the gastrointestinal tract without the dissolution process.

On the other hand, it can be exploited that frequently active substances are present in various polymorphic forms which, although not very different in their pharmaceutical efficacy, but whose solubility is different. Also, the conversion to easily soluble salts can improve solubility in aqueous solutions or the use of co-crystals of the active ingredient.

Another approach to improve the bioavailability of the active ingredients is to enlarge the surface area (A) as well as to improve the solubility (Cₛ) by suitable measures. This can be achieved, for example, by the preparation of solid dispersions using hot melt extrusion or spray-drying technologies or mesoporous silica, wherein the poorly soluble active substance is applied onto an inert carrier. Particularly suitable as supports are biocompatible porous inorganic materials, which are present as corresponding powders with suitable particle sizes. Pharmaceutical scientists developing formulations in industry are able to utilize these three techniques for modifying the physical state of the API, converting the poorly soluble drug from its crystalline form into a stabilized amorphous structure, with significantly enhanced solubility and oral drug absorption.

Spray-drying and hot-melt extrusion are often applied in the manufacture of solid dispersions and solid solutions. This approach dates back to Sekiguchi and Obi, who first introduced eutectic mixtures as a means for solubility enhancement. [Sekiguchi, K. and N. Obi, Man. Chemical & pharmaceutical bulletin, 1961. 9(11): p. 866-872]. In a solid dispersion, the API is generally dispersed or dissolved within a polymeric matrix, either in its crystalline or amorphous state or, in the case of solid and glassy solutions, at a molecular level resulting in a so-called amorphous solid dispersion (ASD). [Dhirendra, K., et al., Pakistan Journal of Pharmaceutical Sciences, 2009. 22(2): p. 234-246]

As mentioned above, theoretically the solubility of a poorly soluble active ingredient can be improved by enlarging the surface area (A) and to minimize the particle size of the drug substance. The micronization method of grinding drug compounds to achieve a smaller particle size is well established. A detailed overview to these methods is given in EP 1 401 401 B1 (ELAN Pharma Int. Ltd. [IE]). In this document a method is disclosed comprising reducing the particle size of a poorly soluble compound to about 1 μm or less using a small-scale mill. The product produced in this process is a dispersion of a nanoparticulate model substance comprising one or more surface stabilizers, which are adsorbed onto the surface of the compound. The reduction in particle size results in an increase of the solubility and/or dispersibility of the compound. Thus, the most important and very widespread method for the production of corresponding nanomaterials is the so-called nano-milling method, which can be carried out using a viscosity enhancer and which is only possible with the addition of stabilizers. As such, however, the grinding process cannot always lead to success, in particular, if the milling process results in the development of electronic charges, which can lead to aggregation of the small particles as large or even larger than the unmilled drug [Lin S.-L.; Menig J.; Leon Lachmann; “Independence of physiological surfactant and drug particle size on the dissolution behavior of water-insoluble drugs”; J. Pharm. Sci. (1968); 57(12); 2143-8].

The second possibility is the bottom-up development by aggregation of smaller particles, e.g. by supercritical precipitation. In the bottom-up development of nanoparticle drug carriers the particulate system is prepared from a state of molecular dispersion type and is allowed to associate with subsequent formation of solid particles. Bottom-up techniques, therefore, seek to arrange smaller components into assemblies of complex structure, e.g. by supercritical precipitation. However, these specific methods for producing high surface area drug particles are not to be considered here, because, among other things, they are very complex and expensive to produce.

One of the well-studied methods for grinding drug particles is wet-milling. In this context, wet-milling using bead mills is one of the most effective ways to decrease the particle size of an API. With this technique, large drug crystals are suspended in a milling medium. The milling medium consists of a fluid containing the milling beads and the API, which must be insoluble in the milling medium. The milling beads need to show more physical robustness than the drug to be nano-milled and must of course be stable against high shear forces in general. To carry out the milling a crude slurry consisting of drug, water and stabilizer is fed into the milling chamber. In the milling chamber, the drug crystals are subject to high energy input provided by the milling medium. The process can be run either in a batch mode or in recirculation. The typical residence time to mill the API down to about 200 nm in mean diameter is in the range between 30 to 60 minutes in batch-mode [E. Merisko-Liversidge, G. G. Liversidge, E. R. Cooper, Nanosizing: a formulation approach for poorly-water-soluble compounds, Eur. J. Pharm. Sci., 18 (2003) 113-120]. Nevertheless, the time-frame needed is drug specific and in other cases it can take hours or even days to achieve the desired size of the drug crystal [F. Kesisoglou, S. Panmai, Y. Wu, Nanosizing—oral formulation development and biopharmaceutical evaluation, Adv Drug Deliv Rev, 59 (2007) 631-644; J. U. Junghanns; R. H. Muller; “Nanocrystal technology, drug delivery and clinical applications”; Int J. Nanomedicine, 3 (2008) 295-309].

The choice of beads used for milling depends on their ability to resist abrasion during the milling process, which would lead to undesired product contamination. Beads made from glass or zirconium are likely to withstand the milling process, but even with these beads potential product contamination by abrasive bead fragments has to be considered carefully.

FIG. 1 shows schematically a possible procedure and an assembly of devices for performing a wet bead nano-milling process. Corresponding facilities for this purpose are commercially available and may even be realized in a single device.

The main disadvantage of nano-milling is that the crystalline API is produced in a liquid state with viscosity enhancer, which leads during the storage to a reduced stability and tendency to recrystallization of the active ingredient. Therefore, stabilization is mandatory. However, the latter is only possible if such materials are prepared by nano-milling in the form of suspensions and only after the addition of stabilizers.

Usually, the next step for producing a dry material from wet-milling suspensions is done by spray drying or freeze drying, spray granulation or even by standard drying in an oven (with or without vacuum). [S. Bose; D. Schenck; I. Ghosh; A. Hollywood; E. Maulit; C. Ruegger “Application of spray granulation for conversion of a nanosuspension into a dry powder form”; European Journal of Pharmaceutical Sciences 47 (2012) 35-43]

### OBJECT OF THE PRESENT INVENTION

As a consequence, the production of nanocrystalline active substance suspensions is much more demanding than the micronization of the active compounds and the preparation of corresponding suspensions, because several challenges come into play when particle size falls below the micrometer range. Conventional milling methods, such as hammer- or jet-milling cannot fulfill the goal of nanosizing due to their construction principles and resulting from physical limitations.

Therefore, there is a need for a suitable preparation method for milling the corresponding APIs into the nanometer range and provide a reproducible particle size. Furthermore, it is further necessary to provide a method by which the produced nanosized drug particles are stabilized. In addition, further processing of API suspensions to a final administration form needs to be done soon after the nano-milling step and such formulations contain all additives needed to produce the nanosized drug particles.

### SUMMARY OF THE INVENTION

The present invention provides a method for producing pharmaceutical compositions with enhanced bioavailability of pharmaceutical active ingredients, in special but not limited to API's which are belonging to the BCS classes II and IV and which in general are poorly soluble drug candidates. The produced pharmaceutical composition of the present invention comprises the pharmaceutical active ingredient of BCS classes II and IV and a pharmaceutically acceptable carrier or excipient and is in the form of solid particles or powder or granules. These solid particles, powder, or granules may further be filled into capsules or compressed, optionally together with additives, to tablets. The present invention further provides a method for preparing the pharmaceutical composition of the present invention, which is characterized by features as given in claims 1-9.

### DETAILED DESCRIPTION OF THE INVENTION

In general, it has been found, that once the particle sizes decrease below one micron, agglomeration or even particle growth by Ostwald ripening may occur, but which has to be avoided. Here, in the context of preparing pharmaceutical formulations, the choice of a suitable stabilizer, i.e. polysorbates or povidones, is crucial for stability requirements of the processed active ingredient. In addition, both, the type of stabilizer and its ratio to drug have to be evaluated empirically. As known from literature the drug to stabilizer ratio on a weight basis usually ranges from 20:1 to 2:1 [Merisko-Liversidge E.; Liversidge G G.; “Nanosizing for oral and parenteral drug delivery: a perspective on formulating poorly-water soluble compounds using wet media milling technology”; Adv Drug Deliv Rev. (2011) 63(6), 427-4054]. A too low ratio will result in agglomeration of particles, while when the ratio is too high in the nano-dispersion small quantities of the comprising drug will already dissolve. This will lead to increased Ostwald ripening due to the imbalance between particle sizes, resulting in redistribution of mass among particles due to their different surface curvatures. The basic scheme of the Ostwald ripening process is that an unequal size distribution between drug particles induces dissolution of smaller particles and the dissolved drug precipitates on larger particles. To prevent this effect, it is important that the production processes result in a narrow particle size distribution.

In principle, there are three main methods to produce stable nanosuspensions of poorly soluble APIs: jet-milling, wet-milling methods using bead mills and high-pressure homogenization.

The wet-milling method has already been mentioned above. In jet-milling a fluid jet mill uses the energy of the fluid (high pressure air) to achieve ultrafine grinding of pharmaceutical powders. In high pressure homogenization (HPH), the solid to be comminuted is first dispersed in a suitable fluid and then forced under pressure through a nanosized aperture valve of a high-pressure homogenizer, which is essentially a bottleneck through which the suspension passes with a high velocity, and then suddenly experiences a sudden pressure drop, turbulent flow conditions and cavitation phenomena.

Nowadays, various pharmaceutical formulations in which the active substances are present in micronized form or as nanoparticles are commercially available for patient use. The following table lists some of the nanoscale products from different suppliers.

Commercialized nanosized drugs are listed in “Advanced Drug Delivery Reviews”. These drugs are prepared using a nanoparticle technology (modified list from [Kesisoglou, F.; Panmai, S.; Wu, Y.; Advanced Drug Delivery Reviews, Volume 59, Issue 7, 30 Jul. 2007, Pages 631-644; “Nanosizing—Oral formulation development and biopharmaceutical evaluation]). This list includes only a selection of products based on nanoparticles but there are further products on the market.

TABLE 1
List of commercialized nanosized drugs

| | Drug | | | Nanoparticle |
|---|---|---|---|---|
| Product | compound | Indication | Company | technology |
| RAPAMUNE ® | Sirolimus | Immunosuppressant | Wyeth | Elan Drug |
| | | | | Delivery |
| | | | | Nanocrystals ® |
| EMEND ® | Aprepitant | Antiemetic | Merck | Elan Drug |
| | | | | Delivery |
| | | | | Nanocrystals ® |
| TriCor ® | Fenofibrate | Treatment of | Abbott | Elan Drug |
| | | hypercholesterolemia | | Delivery |
| | | | | Nanocrystals ® |
| MEGACE ® | Megestrol | Appetite stimulant | PAR | Elan Drug |
| ES | acetate | | Pharmaceutical | Delivery |
| | | | | Nanocrystals ® |
| Triglide ™ | Fenofibrate | Treatment of | First Horizon | SkyePharma |
| | | hypercholesterolemia | Pharmaceutical | IDD ®-P |
| | | | | technology |
| Invega | Paliperidone | Treatment of | Janssen | Elan Drug |
| Sustenna ® | palmitate | Schizophrenia | | Delivery |
| | | | | Nanocrystals ® |

Although the size reduction techniques discussed here are convenient and simple, they are sometimes not suitable and are unfavorable depending upon the types of drug substances and the particles to be micronized or nanosized. Conventional methods of size reduction are often known to have certain typical disadvantages, for example of being less efficient due to a high energy requirement or posing threats because of thermal and chemical degradation of drugs or that end products being not uniform in the particle size distribution. Conventional milling techniques, in particular, are considered to be uncontrolled processes that have limitations in controlling size, shape, morphology, surface properties and electrostatic charge and lead to heterogeneous particle shapes or even agglomerated particles as the end product. To overcome these limitations and to specifically control the particle properties, several particle engineering techniques have been developed and are utilized to produce the required particle size and for carefully controlling the particle properties. As such, different methods of producing micronized or nanosized drug particles were attempted to reduce the particle size of poorly water-soluble drugs to increase their solubility and dissolution, and thus to improve their bioavailability.

One solution to bring nano-milled API into dry stage or into a final formulation is to bring in contact and to combine a nano-milled solution and carrier.

As such, it has been found that in dry stage the material is easier to handle, even in use for direct tableting methods.

Accordingly, nano-milled APIs can be stabilized by depositing the suspension without stabilizer on a carrier.

Therefore, in a first attempt the nano-milled fenofibrate (nanosuspension), which is loaded onto silica particles by freeze-drying technique, is investigated. In this feasibility study two different types of silica materials are tested.

For this, the nanomilled active pharmaceutical ingredient (API) is transferred onto a carrier and moved into dry stage by first preparing a suspension of a solution comprising the API and the particulate carrier and then by freeze drying or standard drying this suspension.

Thus, an oral administration form can easily be established as final formulation by tableting of the received materials on a tablet press, if needed, together with a known binder.

Thus, it has been found, that in its dry stage the material is easier to handle, and even can be used in direct tableting methods. However, it also means that the nano-milled APIs can be easily stabilized by being supported on the carrier without the need of stabilizers, and, in addition to the stabilization, a higher shelf life is achieved for the applied APIs without the addition of stabilizers.

In the following, examples are given showing the unexpected advantageous properties and effects of the invention. APIs used in example are different in their chemical nature (acidic or week base):

In these examples
test APIs are Fenofibrate and Itraconazole
nano-milled API suspensions are prepared with stabilizer
alternative preparations of APIs are prepared without any stabilizer
loading of nano-milled APIs on carrier (e.g. silica) is done by impregnation method
or by
freeze-drying of nano-milled API-silica-suspensions

Samples are stored for comparison and the stability of the suspension after nano-milling is examined.

The term “stable”, as used herein, refers to physical stability, measuring the particle size distribution as described later.

The size reduction of the applied drugs, here of fenofibrate and itraconazole, which are exemplified as sparingly soluble model substances, is achieved by wet-milling of a suspension in an aqueous medium using mechanical means. Preferably the milling is carried out in a suitable ball mill. As described above, the milling also can be proceeded in other suitable mills, provided that therein the particle sizes can be reduced in a desired manner and under suitable conditions. Such a mill can be for example a jet mill, media mill, such as a sand mill, Dyno® mill, or a bead mill. The grinding media in these mills can comprise spherical particles, such as stainless-steel beads or zirconium oxide balls.

As the particle size reduction of the low soluble active ingredients is processed preferably in aqueous dispersion a floating of the ingredient has to be avoided for achieving a reliable grinding result. To stabilize the dispersion, various substances may be added depending on the properties of the active ingredient to be ground. Examples of suitable stabilizers include, but are not limited to gelatin, casein, gum arabicum, stearic acid, calcium stearate, glycerol monostearate, sorbitan esters, macrogel ethers such as cetomacrogel 1000, polyoxyethylene castor oil derivatives, polyoxyethylene sorbitan fatty acid esters such as Tween®, polyoxyethylene stearates, colloidal silicon dioxide, sodium dodecylsulfate, carboxymethylcellulose calcium, carboxymethylcellulose sodium, hydroxyethylcellulose, hydroxypropylcellulose, hydroxypropyl methylcellulose (HPMC), polyvinylpyrrolidone (PVP), poloxamers such as Pluronics® F 68 and F 108, dioctyl sodium sulfosuccinate (DOSS), docusate sodium, sodium lauryl sulfate, Span® 20 and 80, and macrogolglycerol esters such as Cremophor® EL. In combination with the model substances selected here, HPMC (Hypromellose) and DOSS have proven to be particularly suitable additives for viscosity enhancement and as stabilizers.

Nano-milling examples as disclosed in the following are carried out using aqueous dispersions. But depending on the properties of the drug, it may be necessary to carry out the nanomilling in another solvent or solvent mixtures. Examples of suitable liquids include, but are not limited to, water, propylene glycol, dipropylene glycol, polypropylene glycol, ethylene glycol, polyethylene glycol, glycerin, butylene glycol, hexylene glycol, polyoxyethylene and mixtures thereof. Preferably, however, the grinding is carried out in aqueous solution.

In the further course of the preparation of the final active ingredient-containing formulation, it may be necessary that further additives have to be added after the loading of the carrier, such as surfactants or antioxidants, preservatives or tablet adjuvants, like diluents, binders, disintegrants lubricants, glidants. However, in the most preferred embodiment of the present invention, such additives are not required, especially since, when using the silica-based carriers used here in the examples, the free-flowing powders obtained after loading with active ingredient can be pressed directly into tablets. If it should be necessary to add appropriate additives, it is possible for the skilled person to select the suitable ones. The prepared active ingredient-containing formulation is obtained in form of solid particles, as powder or granules, which can be filled into capsules or further processed, if necessary, with tablet adjuvants, and compressed into tablets.

Examples of suitable surfactants include, but are not limited to lecithin, sorbitan monostearate, polysorbates prepared from lauric, palmitic, stearic, and oleic acid, polyoxyethylene monoesters such as polyoxyethyl ethylene monostearate, polyoxyethylene monolaurate, and polyoxyethylene monooleate, dioctyl sodium sulfosuccinate, sodium lauryl sulfate, and poloxamers.

Examples of suitable antioxidants include, but are not limited to, butylated hydroxyl anisole, butylated hydroxyl toluene, tocopherol, ascorbyl palmitate, ascorbic acid, sodium metabisulfite, sodium sulfite, sodium thiosulfate, propyl gallate, and mixtures thereof.

Examples of suitable preservatives include, but are not limited to, methyl paraben, ethyl paraben, propyl paraben, butyl paraben benzoic acid, sodium benzoate, benzyl alcohol, sorbic acid, potassium sorbate, and mixtures thereof.

### LIST OF FIGURES

FIG. 1: shows schematically a possible procedure and an assembly of devices for performing a wet bead nano-milling process.

FIG. 2: shows the DSC curve of FF_29062016_SLC_500_001, plotted next to the DSC curve of pure fenofibrate. The endothermic melting peak of pure, crystalline fenofibrate is clearly visible at about 80° C.

FIG. 3: shows the Comparison of the API releases (loaded silica carriers+fenofibrate), 50 mg API, 1000 mL SGFsp+0.1% SDS, 75 rpm, Mean value [mg/L]+standard deviation [mg/L]

FIG. 4: Results of comparison of formulation achieved using Fenofibrate nanosuspension versus Fenofibrate (crystalline)

FIG. 5: Comparison of results achieved by loading amorphous API in presence of organic solvents (preparation as described before) versus loading by nanosuspension (still crystalline API)

FIG. 6: Release data of the nano-milled particles of loaded Kieselgel SI 5000 batch (FF_29062016_SI_5000_001) are compared with data of loaded nano-milled suspension of third batch of the Parteck® SLC 500 (FF_29062016_SLC_500_003).

FIG. 7: The dissolution of the nano-milled drug without stabilizer applied to a carrier whereby Parteck® SLC 500 and Kieselgel SI 5000 showed a very similar release property with or without stabilizer

FIG. 8: shows the DSC curve of crystalline itraconazole, along with the curves of the nanosuspension-loaded batches of Parteck® SLC 500 and Kieselgel SI 5000

FIG. 9: shows the results of the batch, ICZ_16092016_SLC_2, which releases with a maximum concentration of approx. 3 mg/L and substantial faster than itraconazole crystalline sample compared.

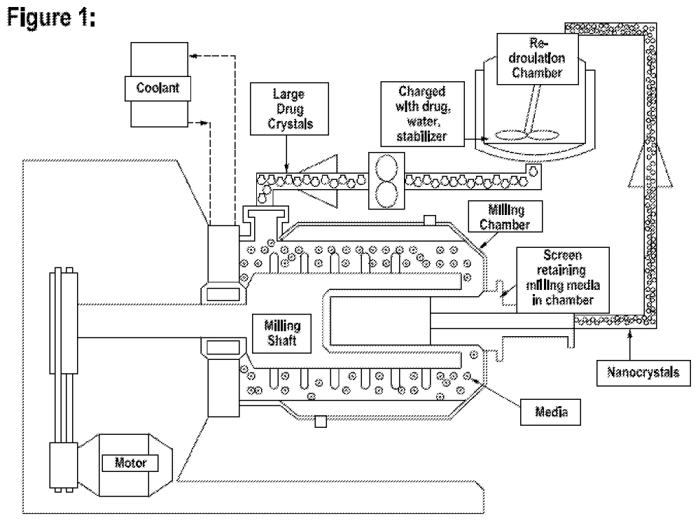

In the following the present invention is shown by different experiments and examples. The results of these experiments are explained in detail, discussed and evaluated. These additional embodiments illustrate the general applicability of the principle of the invention and, therefore, as well as the examples, are included in the disclosure of the present invention.

### Examples

The present description enables the person skilled in the art to apply the invention comprehensively. Even without further comments, it is assumed that a person skilled in the art will be able to utilize the above description in the broadest scope.

Practitioners will be able, with routine laboratory work, using the teachings herein, to prepare active ingredients comprising formulations as defined above in the new process.

The invention described may be further illustrated by the following examples, which are for illustrative purposes only and should not be construed as limiting the scope of the invention in anyway.

If anything is still unclear, it is understood that the publications and patent literature cited should be consulted. Accordingly, these documents are regarded as part of the disclosure content of the present description.

For better understanding and in order to illustrate the invention, examples are given below which are within the scope of protection of the present invention. These examples also serve to illustrate possible variants. Owing to the general validity of the inventive principle described, however, the examples are not suitable for reducing the scope of protection of the present application to these alone.

Furthermore, it goes without saying to the person skilled in the art that, both in the examples given and also in the remainder of the description, the component amounts present in the compositions always only add up to 100% by weight, volume or mol-%, based on the composition as a whole, and cannot exceed this, even if higher values could arise from the percent ranges indicated. Unless indicated otherwise, % data are % by weight, volume or mol-%, with the exception of ratios.

The temperatures given in the examples and the description as well as in the claims are always in ° C.

Methods (See in the Following Text):

Loss on drying
Differential Scanning Calorimetry
Release
Determination of content by HPLC
Determination of content by H₁-NMR
Light micrographs
(only for FF_2906_SLC_500_001)

Drying Loss (IR Balance)
Device: Mettler PM 400; Mettler LP16 (Mettler Toledo GmbH, Gießen, Germany)
Weighing: 0.3 g (Minimum)
Temperature: 105° C.
Method: 0-100%
Constance: 1 Digit/10 s
Aluminum dish: ME-13865
No. of determinations: 3

The loss on drying should ideally be below 1% for release. If the drying loss is higher, it may be necessary to dry again.

Light Micrographs
Device: Light Microscope Zeiss Stemi 2000-C (Carl Zeiss AG, Oberkochen, Germany)
Camera Power Shot A640 (Canon Germany GmbH, Krefeld, Germany
Cold light lamp CL1500 ECO (Carl Zeiss AG, Oberkochen, Germany)
Measuring Software: Axio Vision Rel. 4.8 (Carl Zeiss AG, Oberkochen, Germany)
Slides: 76×26×1 mm; ISO 8037/1; edges 90° ground, pre-cleaned, without mat edge
(Paul Marienfeld GmbH & Co. KG, Lauda-Königshofen, Germany)

The substance to be measured is evenly distributed on the slide and the lighting conditions and sharpness adjusted until the desired display is achieved.

Differential Scanning Calorimetry
Device: Mettler Toledo DSC 3+ (Mettler Toledo, Gießen, Germany),
STARe-Excellence-Software (Mettler Toledo, Gießen, Germany)
Weighed quantity: 2-4 mg for 40 μL aluminum crucible
30-40 mg for 100 μL aluminum crucible
Atmosphere: 50.0 mL/min N₂
Temperature range: 25-350° C.
Heating rate: see in the following
No. of determinations: at least 2

Type of Heating:

Program 1: (“40-100/5K”)

Continuous heating of the sample from 30° C. to 120° C. with a heating rate of 5 K/min.

Program 2: (“40-100/30K”)

Continuous heating of the sample from 30° C. to 120° C. with a heating rate of 30 K/min.

Program 3: (“40-60/10K_60iso_5min_60-90/2K_Alu40_N2”)

Continuous heating of the sample from 40° C. to 90° C. with a heating rate of 2 K/min including temperature maintenance phase of 5 min at 60° C.

Program 4: (“25-100/5K_(Alu100_N-2)”)

Continuous heating of the sample from 25° C. to 100° C. with a heating rate of 5 K/min.

Program 5: (“25-85/5K_85_5min_85-50/5K_(Alu100_N-2)”)

Continuous heating of the sample from 25° C. to 85° C. with a heating rate of 5 K/min, keeping the temperature for 5 min, cooling the sample from 85° C. to 50° C. with a cooling rate of 5 K/min.

Release of Active Ingredient (Sotax 1 and 2)
Device: Sotax 1 and 2,
Release apparatus: Sotax AT 7smart (Sotax AG, Lörrach, Germany),
Photometer Agilent 8453 (Agilent Technologies, Waldbronn, Germany)
Number of vessels: 3 or 6
Method: Paddle
Medium: SGFsp+0.1% sodium dodecyl sulfate
Amount of medium: 1000 mL
Temperature of medium: 37° C.
Rotation: 75 rpm
Duration: 2 h
Time of sampling: 5, 10, 15, 20, 25, 30, 45, 60, 75, 90, 105, 120 min
Final spin: no
Cuvette layer thickness: 5 mm
Wavelength: 288 nm
Dose of active ingredient: 50 mg
Drug loading: about 16%
Filter removal station: GF/D 2.7 μm
Sample volume: 2.5 mL

Each sample is collected in a test tube with the automatic sampler. The samples are then measured offline by HPLC determination (see Method Determination of content by HPLC).

Determination of Content by HPLC
Device: HPLC-system LaChrom® Elite (Hitachi Europe GmbH, Düsseldorf, Germany)
Detector: UV Detector L-2400 VWR Hitachi (Hitachi Europe GmbH, Düsseldorf, Germany)
Autosampler: Autosampler L-2200 VWR Hitachi (Hitachi Europe GmbH, Düsseldorf, Germany)
Column: LiChroCART® 125-4, LiChrospher® 100 RP-18e (5 μm)
Eluent: Acetonitrile/Milli-Q water/trifluoroacetic acid (700:300:1)
Wash solution sampler: Acetonitrile/Milli-Q-Wasser (1:1)
Column oven—temperature: 50° C.
Injection volume: 25 μL
Wave length—detector: 288 nm
Flow rate: 2.0 mL/min (isocratic)
Duration—run: 5 min
Sequence: XXXXXXXX_01_PK_Fenofibrate_Disso_Nanosuspensionen 1. Seq
Method: XXXXXXXX_01_PK_Fenofibrate_Method
Filter—sample preparation: Whatman™ Anotop™ 10, 0.02 μm, Cat.-No.: 6809-1002

Prior to filling into vials, each sample from the release is first filtered with a syringe with Luer-Lock connection and above filters for sample preparation to retain any particles of the nanosuspension and eliminate a systematic error. Such a systematic error can be found in majority of scientific papers and patents as most evaluation do not carefully remove still nano-milled particle from the samples by using appropriate filters. Only soluble API content should be detected.

For evaluation of the HPLC results, the saturation concentration is determined by fixed lab-method and the release of crystalline fenofibrate from a lab test done before (online determination) is taken.

Determination of Content by H₁-NMR
Method: 1H-NMR spectroscopy
Condition: DMSO-d6
Measurement mode: content [%]

The measurement is made by an external analysis order. For this purpose, a sample tube is filled up to half and sent to the appropriate place. The result is given in % content.

Particle Size Determination (Zetasizer Nano ZS)
Device: Zetasizer Nano ZS (Malvern Instruments Ltd, Herrenberg, Germany)
Amount: few drops (diluted, non-turbid solution)
Dispersing medium: desalinated water (viscosity: 0.8872 cP)
Measuring range: 0.3 nm-10 μm
Measuring time some minutes
Temperature: 25° C.
Equilibration time: 60 s
Number of measurements: 6×12 measurements
Method of measurement: Size measurement (Number)
Measurement angle: 173° Backscatter (NIBS default)
Active ingredient: Fenofibrate (RI: 1,547*; Absorption: 0.01)
Data processing: general purpose
Type of cuvettes: DTS0012—Disposable sizing cuvette
Cuvette: 10×10×45 mm Polystyrol/Polystyrene (REF: 67.754; Sarstedt AG & Co, Numbrecht, Germany)
Evaluation: Formation of an average of at least 3 determinations
(*source: http://www.lookchem.com/Fenofibrate/)

The sample is filled into a cuvette (preferably 40 μL cuvette) up to the mark of the Zetasizer and measured. If the results are not “good” (see “Expert Advise”), repeat the measurement with a more dilute sample. The sample should be slightly cloudy at most, in order to exploit the optimal working range of the Zetasizer.

General Information “Nano-Milling”

Wet-Milling:

The milling is to be carried out with the Dyno®-Mill Research Lab (Willy A. Bachofen Maschinenfabrik, Muttenz, Switzerland)
Filling volume: 60-200 mL
Grinding balls: SiLi ZYP 0.2-0.3 mm
Weighing of grinding balls: 200 g
Stirring speed: 2000-4000 rpm
Temperature of cooling liquid: −10° C.

Loading of Two Carrier Parteck® SLC 500/Kieselgel SI 5000 with Different Pore Sizes
Devices: Heidolph RZR 2102 control Laboratory stirrer (Heidolph Instruments, Schwalbach, Germany) Head stirrer with stirring blade
Weighing (silica): approx. 10 g
Weighing (suspension): approx. 10 g (and approx. 20 g)
Stirring speed: 70 rpm
Type of cannula: 0.80×120 mm BL/LB
Dosing speed: approx. 2 g/min

Parteck® SLC 500 or Kieselgel SI 5000 is loaded by uniform application of the nanosuspension using a 10 mL syringe with Luer-Lock cap and cannula. The carrier material is in a beaker in which the stirrer fits straight into it. During application, stirring is continued with the stirrer. If the mixing of the carrier material is not complete, the height and immersion depth of the stirrer can be changed manually (for example, by lifting/lowering of the beaker).

Other Devices:
Magnetic stirrer: IKA®-Werke GmbH & Co. KG, Staufen, Germany

TABLE 1
Materials:

| Material | Origin |
|---|---|
| Fenofibrate | BEC Chemicals Provate Ltd., Ind. |
| Hypromellose (HPMC) (Pharmacoat | Shin Etsu Chemical Co., Ltd. |
| 603) | |
| Dioctylsulfosuccinate-Natrium = | Aldrich Chemistry |
| DOSS | |
| Parteck ® SLC 500 | Merck KGaA, Darmstadt |
| Kieselgel SI 5000 | Merck KGaA, Darmstadt |
| | (synthesis) |
| Milli-Q-Wasser | Merck KGaA, Darmstadt |

The Parteck® SLC 500 is a silica gel with a specific surface area of 500 m²/g (BET measurement) and an average pore size of 6 nm.

The Kieselgel SI 5000 comes from a silica synthesis of Merck KGaA by using addition and melting of NaCl to change pore size of the carrier. It has a specific surface area of 3 m²/g (BET measurement) and has an average pore size of 500 nm.

To stabilize the nanosuspension, HPMC and DOSS are added to the suspension medium. Without these stabilizing agents, the fenofibrate nanosuspension produced might be prone to rapid formation of aggregates and build-up of larger particles due to greatly increased surface effects, such as electrostatic attraction and dissolution rate (Ostwald ripening).
SGFsp: simulated gastric fluid sine pepsin
SDS: sodium dodecyl sulfate

Reasons for the Experiments:

The aim of the following experiments is to find out whether an improvement in the release of the sparingly soluble active ingredient is achieved by a impregnation loading method when the active ingredient is applied in the form of a nanosuspension where the API is suspended as nano-particles but still in crystallin state. The carrier used for this purpose is Parteck® SLC 500 and fenofibrate as the active ingredient.

In addition, Kieselgel SI 5000 is used as support material and loaded with the crystalline fenofibrate nanosuspension. Here, the influence of the pore size on the release of fenofibrate is investigated.

Most important during HPLC analyzes is the appropriate filtration of samples. Prior to filling into vials, each sample from the release is first filtered with a syringe with Luer-Lock connection and above filters for sample preparation to retain any particles of the nanosuspension and eliminate a systematic error. Such a systematic error can be found in majority of scientific papers and patents as most evaluation do not carefully remove still nano-milled particle from the samples by using appropriate filters. Only soluble API content should be detected.

Carrying Out the Experiments:

At the beginning of the experiments, a fenofibrate suspension (see Method of Experiment 1 A) is prepared which is stabilized with HPMC and DOSS (dioctylsulfosuccinate sodium) and then nanomilled (see Methods Nano-milling in the following). The nanosuspension obtained is stored in the refrigerator at temperatures between 2 and 8° C.

Using the impregnation method (as described in the following examples), the carriers Parteck® SLC 500 and Kieselgel SI 5000 are loaded in a ratio of 1:1 or 2:1 (w/w) with the fenofibrate nanosuspension finding out best loading ratio but to see if higher loading is possible as well without impact of loading amount. The drying is then carried out by freeze-drying (see Method “Nano-milling” in the following). Since Parteck® SLC 500 has hygroscopic properties, all batches produced are stored in the desiccator over orange gel.

TABLE 2
Overview of the experiments for dissolution
measurement and comparative experiments

| | | Active | |
|---|---|---|---|
| Experiment | Carrier | ingredient | Comments |
| FF_2906 | — | Fenofibrate | Fenofibrate- |
| | | | Nanosuspension |
| FF_2906_SLC_500_001 | Parteck ® | Fenofibrate | |
| | SLC 500 | | |
| FF_2906_SLC_500_002 | Parteck ® | Fenofibrate | |
| | SLC 500 | | |
| FF_2906_SLC_500_003 | Parteck ® | Fenofibrate | |
| | SLC 500 | | |
| FF_2906_SI_5000_001 | Kieselgel | Fenofibrate | |
| | SI 5000 | | |

Method

Experiment 1

A) Preparation of the Suspension

5 g of HPMC and 0.2 g of DOSS are placed in a beaker and are dissolved in 154.88 g of deionized water by stirring with a magnetic stirrer for about 40 minutes. When the substances are dissolved, 80.0 g of the received solution are placed in a beaker and 20 g of fenofibrate are added and suspended in the solution by stirring (450 rpm) for 10 minutes.
Theoretical fenofibrate content: 20% (w/w)

A′) Determination of the Saturation Concentration (for Analytical Measurement)

For the preparation of the saturated solution, the magnetic stirrer of IKA®—Werke GmbH & Co. KG, Staufen, Germany, is also used here.

For the preparation of the saturated fenofibrate solution, which is needed to determine the saturation concentration, 1 tablespoon of fenofibrate is suspended in 200 ml of solution (SGFsp+1% SDS). This suspension is heated to a temperature of 40° C. and stirred at 300 rpm for at least 24 hours, here for 72 hours. The beaker is covered with Parafilm during this time. Subsequently, the saturation concentration is measured by HPLC determination.

Method

Nano-Milling

The suspension (Experiment 1 A) is filled into the hopper of the mill and the milling process is started at 2000 rpm. The particle size is checked every 5 minutes (see some results of every 15 minutes/Table 3) via Dynamic Light Scattering. If necessary, the stirring speed can be increased to 3000 or 4000 rpm. Overall, the milling process should not exceed a time of 2 hours.

TABLE 3
Particle size of nano-milled fenofibrate as function on milling time

| Time | d(10) | d(50) | d(90) | Median |
|---|---|---|---|---|
| [min] | [μm] | [μm] | [μm] | [μm] |
| 0 | 27.2 | 101.5 | 267.1 | 94.9 |
| 15 | 0.088 | 0.150 | 1.029 | 0.158 |
| 30 | 0.079 | 0.131 | 0.206 | 0.129 |
| 45 | 0.077 | 0.131 | 0.198 | 0.125 |
| 60 | 0.076 | 0.131 | 0.189 | 0.120 |

B) Applying the Active Ingredient on to the Silica Carrier
a) approx. 10 g of suspension are applied to 10 g of Parteck® SLC500 with stirring, resulting in a loading of about 16.4%.
b) approx. 20 g of suspension are applied to 10 g of Parteck® SLC500 with stirring, resulting in a loading of about 27.5%.

C) Freeze-Drying of Resulting Loaded Carriers
Device: Freeze Dryer Gamma 2-16 LSC (Christ Gefriertrocknungsanlagen Martin Christ, Osterode, Germany)
Cooling: water cooling

The loaded, moist products from a) and b) are freeze-dried under the following conditions in a beaker:

TABLE 4
a) Program 1: “Nano-milling”

| | |
|---|---|
| Temperature | Time |
| [° C.] | [h] |

| | | |
|---|---|---|
| Freezing | −45° C. to −35° C. | ~6 |

| | | | |
|---|---|---|---|
| First drying | −35° | C. | ~25 |
| Second drying | 0° | C. | 1 |
| | +25° | C. | 18 |
| Total | | | 50 |

TABLE 5
b) Program 2: “Nanosus PK”

| | |
|---|---|
| Temperature | Time |
| [° C.] | [h] |

| | | | |
|---|---|---|---|
| Freezing | −45° | C. | 8 |
| Main drying | −35° | C. | 30 |
| Second drying | 0° | C. | 4 |
| | +25° | C. | 18 |

| | |
|---|---|
| Total | 60 |

The samples are placed into the freeze dryer for freeze drying and the freeze dryer is closed.

Water is used for cooling (first the drain is turned on, only then the inlet!). Then the desired program is started. After freeze-drying, the drying loss of the product should be determined as described. If drying is insufficient, further drying is carried out

D) Analysis of the Product Obtained
a) DSC/XRD studies: check of the physical state of the API
b) HPLC/NMR studies: determination of the drug content on the silica
c) release->offline, samples over 0.2 μm PTFE filter (comparison with nanosuspension before adding silica)
d) stability study

TABLE 6
The batches are produced with the following amounts:

| | | Amount of | Theor. |
|---|---|---|---|
| | Amount of | suspension | content |
| Batch no. | Silica [g] | [g] | [%] |
| FF_29062016_SLC_500_001 | 10.00 | 10.00 | 16.6 |
| FF_29062016_SLC_500_001_a | 10.00 | 20.00 | 28.6 |
| FF_29062016_SLC_500_002 | 10.06 | 9.84 | 15.7 |
| FF_29062016_SLC_500_002_a | 10.00 | 20.00 | 28.6 |
| FF_29062016_SLC_500_003 | 10.00 | 9.93 | 16.6 |
| FF_29062016_SLC_500_003_a | 10.00 | 20.00 | 28.6 |
| FF_29062016_SI_5000_001 | 9.99 | 9.87 | 16.5 |
| FF_29062016_SI_5000_001_a | 10.00 | 20.00 | 28.6 |

Experiment 2

(Nano-Milling without Viscosity Enhancer)
A) Preparation of the suspension

0.1 g of DOSS is dissolved in 79.9 mL of deionized water. When the substance is dissolved, 20 grams of fenofibrate are suspended in the solution. The suspension is filled into the hopper of the mill and the milling process is started at 2000 rpm. The particle size is checked every 5 minutes via Dynamic Light Scattering. If necessary, the stirring speed can be increased to 3000 or 4000 rpm. Overall, the milling process should not exceed a time of 2 hours.
B) Applying the active ingredient to the silica carrier
a) approx. 10 g of suspension are applied to 10 g of Parteck® SLC500 with stirring, resulting in a loading of about 16.6%.
b) approx. 20 g of suspension are applied to 10 g of Parteck® SLC500 with stirring, resulting in a loading of about 28.6%.
C) Freeze-drying of resulting loaded carriers
   The loaded, moist products from a) and b) are freeze-dried under the same conditions as in Experiment 1.
D) The analytical evaluation of the products obtained is carried out in the same manner as in Example 1.

Experiment 3
(without viscosity enhancer and without stabilizer)
A) Preparation of the suspension
   20 g of fenofibrate are suspended in 80 mL of deionized water. The suspension is filled into the hopper of the mill and the milling process is started at 2000 rpm. The particle size is checked every 5 minutes via Dynamic Light Scattering. If necessary, the stirring speed can be increased to 3000 or 4000 rpm. Overall, the milling process should not exceed a time of 2 hours.
B) Applying the active ingredient to the silica carrier
a) approx. 10 g of suspension are applied to 10 g of Parteck® SLC500 with stirring, resulting in a loading of about 16.6%.
b) approx. 20 g of suspension are applied to 10 g of Parteck® SLC500 with stirring, resulting in a loading of about 28.6%.
C) Freeze-drying of resulting loaded carriers
   The loaded, moist products from a) and b) are freeze-dried under the same conditions as in Experiment 1.
D) The analytical evaluation of the products obtained is carried out in the same manner as in Example 1.

Evaluation of the Experiments:

Assessment and comparison of the results obtained

FF_29062016_SLC_500_001:

This batch is dried according to Program 1 “Nano-milling” as described above. Since there was a disruption during the drying over the weekend, the program was canceled after 66 hours in the main drying.

The drying loss is on average at about −0.99%.

FF_29062016_SLC_500_002/FF_29062016_SI_5000_001:

These batches have been dried together in Program 2 “Nanosus_PK”.

The drying loss after freeze-drying is on average for FF_29062016_SLC_500_002
−3%

and for

FF_29062016_SI_5000_001 −0.13%.

FF_29062016_SLC_500_003:

This batch has been dried in Program 1 “Nano-milling”. Since the drying loss (n=1) according to the program is −12.98%, the batch is dried once more in Program 2 “Nanosus_PK”.

The drying loss after drying is −1.01%.

Results: Optical Assessment

FF_29062016_SLC_500_001

After loading of Parteck® SLC 500 with the fenofibrate nanosuspension, the support material is slightly clumped. After freeze-drying, these lumps remain. But they are easy to crush with a spatula. An influencing factor in this context may be the metering rate during loading. Since by manual dosing, fluctuations in the dosing rate can occur here. The remainder is loose powder which is of fine consistency.

FF_29062016_SLC_500_002

Also, in the second batch the loading of Parteck® SLC 500 with fenofibrate nanosuspension leads to the formation of smaller lumps. However, they are smaller in relation to those of the first batch. These lumps also can be easily crushed with a spatula.

FF_29062016_SLC_500_003

As well as in the other batches of the loading of Parteck® SLC 500, some clumps are formed after the impregnation, which can easily be crushed with a spatula. Under the light microscope, all nano-milled loaded Parteck® SLC 500 particles are only recognizable as blurred structures. The applied nanosuspension is neither recognizable nor visible fenofibrate crystals have been formed prove successful loading of nano-milled particles distributed on carrier.

FF_29062016_SI_5000_001

Comparable as during loading of Parteck® SLC 500, the loading of the Kieselgel SI 5000 produces some lumps which may have different sizes. But in comparison to loaded Parteck® SLC 500, here the remaining loose powder is floury-like_

FF_29062016_SLC_500_001_a and Further Experiments (=20 g Loadings)

The optical assessment of FF_29062016_SLC_500_001_a and the further samples loaded with 20 g nano-milled suspension for comparison reasons, results in similar powder properties as 10 g loadings. Materials are partly clumped together, but samples were easy to transfer to flowable powder important for further processes as tableting. Based on the optical assessment we follow up with the analytical evaluation of the materials loaded with 10 g suspension only.

Particle Size Distribution of the Nanosuspension

As described above, the particle size distribution is measured with: Zetasizer Nano SZ. Samples are stored for comparison and the stability of the suspension after nano-milling is examined (Table 7).

TABLE 7

| | Process | 2 weeks | 10 weeks |
|---|---|---|---|
| | (t₀) | (t₁) | (t₂) |
| Mean [nm] | 126.94 | 128.35 | 142.20 |
| Std. Dev. [nm] | 15.70 | 16.10 | 40.71 |
| d(10) [nm] | 105.70 | 106.40 | 56.20 |
| d(50) [nm] | 126.00 | 127.50 | 129.00 |
| d(90) [nm] | 147.80 | 149.00 | 236.00 |

Due to the greatly increased specific surface of the particles of the nanosuspension, the solution processes may accelerate in this suspension, so that the fenofibrate dissolves faster. This can lead to the growth of larger particles, while smaller particles completely dissolve. This effect is called Ostwald ripening.

But in this experiment, the effect is small. This means the growth of suspension crystals is slow and could be seen after 10 weeks storage. Within 2 weeks they grow on average within acceptable range to use the nanosuspension over a longer period. So, the nanosuspension is sufficiently well stabilized by the use of HPMC and DOSS.

Determination of Content by H₁-NMR

TABLE 8
The H₁-NMR-API content evaluation showed following values:

| | Theoretical | Measured |
|---|---|---|
| | content | content |
| batch | [%] | [%] |
| FF_29062016_SLC_500_001 | 16.6 | 13.6 |
| FF_29062016_SLC_500_002 | 15.7 | 13.5 |
| FF_29062016_SLC_500_003 | 16.6 | 13.0 |
| FF_29062016_SI_5000_001 | 16.5 | 15.0 |

The actual measured content is up to 3.5% below the theoretical content. There may be many reasons for this deviation: for example, already during nano-milling a decrease in fenofibrate content may occur when the suspension is transferred to the nanomill. When transfer is done always a small remainder suspension stays in the transport vessel. It is possible that, despite shaking, a certain amount of fenofibrate crystals has settled there, which remain in the vessel during pouring. Since all batches have a reduced content, it is probably a systematic error.

Differential Scanning Calorimetry (Evaluation of API Morphology)

(DSC Measurements)

TABLE 9
Evaluation if amorphous/crystalline morphology:

| | | amorphous/ |
|---|---|---|
| batch | carrier | crystalline |
| FF_29062016_SLC_500_001 | Parteck ® SLC 500 | crystalline |
| FF_29062016_SLC_500_002 | Parteck ® SLC 500 | crystalline |
| FF_29062016_SLC_500_003 | Parteck ® SLC 500 | crystalline |
| FF_29062016_SI_5000_001 | Kieselgel SI 5000 | crystalline |

The referring results are to be found under the corresponding sub-items for the DSC measurement.

Identification of the Melting Peaks of Fenofibrate

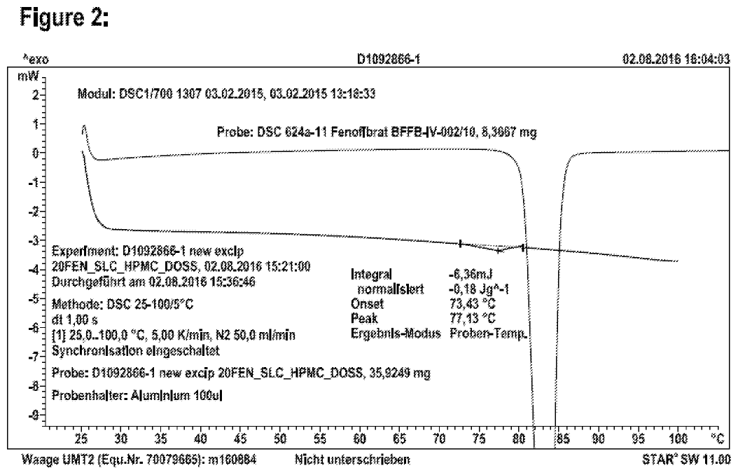

FIG. 2 shows the DSC curve of FF_29062016_SLC_500_001, plotted next to the DSC curve of pure fenofibrate. The endothermic melting peak of pure, crystalline fenofibrate is clearly visible at about 80° C. It can be seen that the melting peak of the fenofibrate nanosuspension on Parteck® SLC 500 is significantly less pronounced and shifted to lower temperature. While the fenofibrate peak of pure fenofibrate sets in sharply, the charged Parteck® SLC 500 is more likely to have only a “dent” in the curve. It is still crystalline on the Parteck® SLC 500.

The DSC curve of FF_29062016_SLC_500_001 also shows a slight melting point depression of the fenofibrate. Since there is not pure fenofibrate in the sample, both the hydroxypropylmethyl cellulose used and also the DOSS can lower the melting point.

In addition, a “masking” of the heat transfer by the Parteck® SLC 500 could take place, so that a defined, clear melting peak is concealed.

Dissolution Measurements: Batches of Loaded Parteck® SLC 500

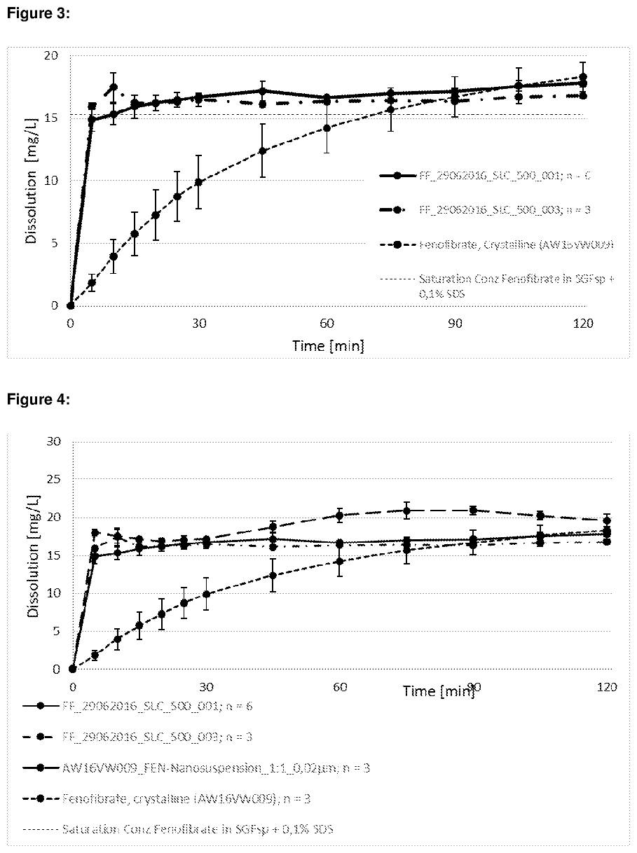

FIG. 3 shows the Comparison of the API releases (loaded silica carriers+fenofibrate), 50 mg API, 1000 mL SGFsp+0.1% SDS, 75 rpm, Mean value [mg/L]+standard deviation [mg/L].

In the release study, the Parteck® SLC 500 batches 1 and 3 show that the fenofibrate nanosuspensions release the active substance comparably well. Both achieve the saturation concentration of approx. 15 mg/L after only 5 minutes, which is significantly faster as it is by dissolving pure crystalline fenofibrate. Crystalline fenofibrate reaches the saturation concentration after 60 minutes. Overall, the saturation concentration is only slightly exceeded with the nano-suspension of Parteck® SLC 500, but also with the crystalline active ingredient. As expected, (as the morphology of API was measured to be still crystalline and not amorphous) there is no improvement in the solubility by nanoparticulate API loaded Parteck® SLC 500 compared to fenofibrate not loaded on a carrier. Very much favorable in comparison between pure API and nanosuspension loaded on carrier is that the loaded Parteck® SLC 500 batches have a greatly increased dissolution rate in the beginning.

Comparison of Results Achieved Using Fenofibrate Nanosuspension Versus Fenofibrate (Crystalline) is Shown in FIG. 4.

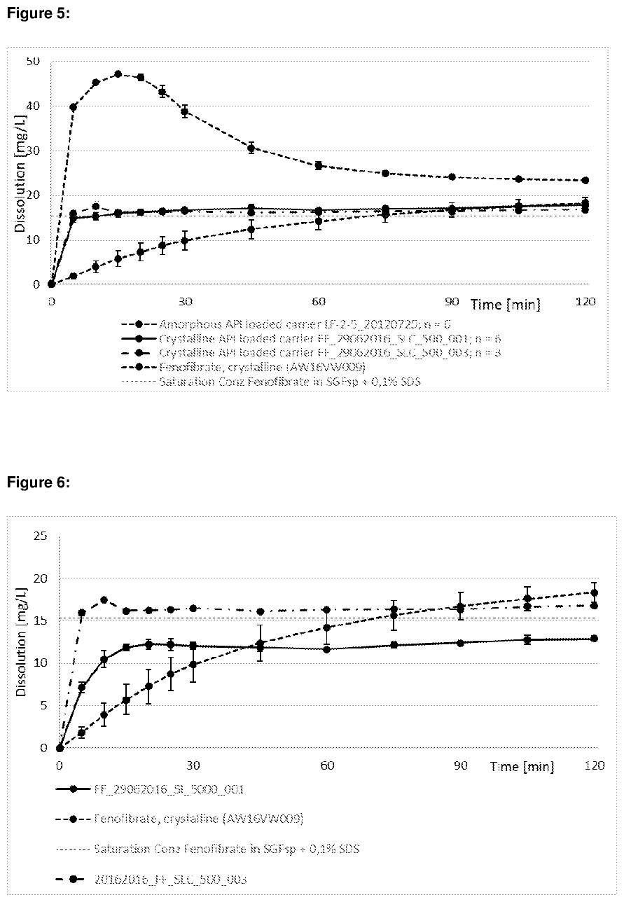

Comparison of Results Achieved by Loading Amorphous API in Presence of Organic Solvents (Preparation as Described Before) Versus Loading by Nanosuspension (Still Crystalline API) (FIG. 5)

In contrast to the Parteck® SLC 500, which is loaded with a nanosuspension (and API is due to physical milling still crystalline), the release of organically loaded Parteck® SLC 500 (API is loaded in amorphous morphology) shows a significantly higher, initial increase in concentration (so-called supersaturation). This reaches its maximum after 15 minutes at about 47 mg/L. This is followed by a decrease in concentration with asymptotic approximation to 25 mg/L after 90 minutes.

Compared to the organic loading, the two samples nanosuspension loaded Parteck® SLC 500 reaches its maximum concentration in 5 minutes after release. After reaching the maximum, the concentration remains constant, in contrast to organic loading; this maximum is slightly above the saturation concentration of the fenofibrate.

The maximum concentration of the organically loaded Parteck® SLC 500 was 47 mg/L; the highest released concentration in nanoparticulate loaded Parteck® SLC 500 was only 25 mg/L. Positive in this context, however, is the lack of recrystallization with a decrease in the concentration of nano-milled fenofibrate loaded, Parteck® SLC 500.

Systematic error can be found in majority of scientific papers and patents as most evaluation do not carefully remove during analytical evaluation still nano-milled particle from the samples by using appropriate filters. Only soluble API content should be detected otherwise API concentration detected above solubility of API in crystalline state and wrong conclusions are based on often.

In FIG. 6 the releases of the nano-milled particle loaded Kieselgel SI 5000 batch (FF_29062016_SI_5000_001) are compared with the same amount of nano-milled suspension loaded third batch of the Parteck® SLC 500 (FF_29062016_SLC_500_003).

Conclusions from these Comparative Measurements

The aim of the experiment was to verify the release of the model drug fenofibrate by nanomilling and subsequent loading of the suspension onto Parteck® SLC 500 (app. 6 nm pore diameter measured) and compare it's dissolution properties versus with the same procedure loaded Kieselgel SI 5000 carrier, with pore diameter in the range of 500 nm.

It was found by this experiment, that the use of a fenofibrate nanosuspension which is applied onto a Kieselgel SI 5000 support, has a significantly faster dissolution rate than crystalline, micronized fenofibrate. A supersaturation or faster dissolution is not observed in comparison to loaded Parteck® SLC 500 even a little smaller total dissolution could be measure using the Kieselgel SI 5000 loaded sample.

The stabilization and release of the API seems to result from the surface and pore nature of the Parteck® SLC 500 as well as Kieselgel SI 5000 and not from the pore diameter.

Further experiments and measurements must confirm these results, for example with itraconazole.

Further experiments without Addition of Stabilizers

In previous studies HPMC and DOSS have been used as a stabilizer and viscosity enhancer for nanosuspension production. In order to test if favorable material of nano-milled loaded carrier, without additional stabilizer is possible to prepare in order to be able to use such not so complex materials in final administration forms, the nanosuspension is loaded onto Parteck® SLC 500 and Kieselgel SI 5000 and the release profile is investigated.

As previously described, a fenofibrate suspension is prepared but without the addition of DOSS as stabilizer. The resulting suspension is then nanomilled. The nanosuspension obtained is stored in the refrigerator at a temperature between 2 and 8° C.

The carriers Parteck® SLC 500 and Kieselgel SI 5000 are each loaded in a ratio of 1:1 (w/w) with the fenofibrate nanosuspension using the impregnation method. Subsequently, the drying is carried out by freeze-drying. Since Parteck® SLC 500 has hygroscopic properties, all batches produced are stored in the desiccator over orange gel.

Then the release of the active ingredient from the samples and the loss on drying of the samples is determined.

For these measurements, samples are prepared with a theoretical fenofibrate content of approximately 18.0% (w/w).

The loading of the carriers is carried out as described in “Loading of Parteck® SLC 500/Kieselgel SI 5000”.

Here, three loadings were made with Parteck® SLC 500 and one Kieselgel Si 5000 loading, always using the prepared nanosuspension without addition of stabilizer (Table 10).

The batches are produced with the following weights:

TABLE 10

| | Silica | Suspension |
|---|---|---|
| batch | [g] | [g] |
| FF_HPMC_SLC_1 | 10.00 | 10.09 |
| FF_HPMC_SLC_2 | 10.02 | 9.99 |
| FF_HPMC_SLC_3 | 10.00 | 10.06 |
| FF_HPMC_SI_{—} | 10.04 | 9.99 |

Results:

Preparation of Nanosuspension without Stabilizers

The preparation of a nanosuspension, without stabilizers DOSS or viscosity enhancer HPMC, only with fenofibrate and MilliQ water is only with a short milling time possible thus commercial process may not feasible to establish. It comes to the floating of the drug and the mill clogged. The addition of HPMC (hypromellose=Pharmacoat 603) allows nano-milling even the suspension foams a little bit more as with addition of DOSS.

Weighing's for Nanomilling without any Stabilizers:
20.03 g Fenofibrate
80.0 g MilliQ water

Weighing's for Nanomilling with HPMC:
19.99 g Fenofibrate
2.52 g HPMC/Pharmacoat 603
77.51 g MilliQ water

Drying Loss

TABLE 11

| | loss on drying |
|---|---|
| batch | [% w/w] |
| FF_HPMC_SLC_1 | 2.15% |
| FF_HPMC_SLC_2 | 1.98% |
| FF_HPMC_SLC_3 | 1.06% |
| FF_HPMC_SI_1 | 1.12% |

The drying loss of the samples is just over 1%. The drying loss of the samples was not determined directly after freeze-drying, but only a few days later. Therefore, it can be assumed that despite the storage in the desiccator, the dry loss has increased slightly. Since the values are under 3% self imposed mark, no subsequent drying of the samples was carried out.

Release of Nano-Milled Fenofibrate without Stabilizer Loaded on Carrier

The dissolution of the nano-milled drug without stabilizer applied to a carrier is better compared to the crystalline drug (not milled). Fenofibrate nano-milled (without stabilizer) loaded Parteck® SLC 500 and Kieselgel SI 5000 as carrier enables a faster release as the pure crystalline drug. The use of formulation without stabilizers in final administration forms as tablets or capsules is favorable, as no additional influence or interference of stabilizer with API has to be considered during the development or clinical phases. API nano-milled formulations reported so far are containing stabilizer resulting in more complex administration forms without easy prediction of influence of additives.

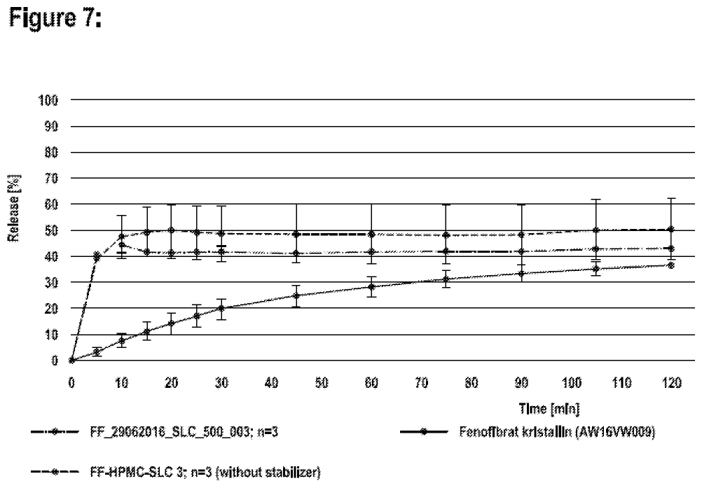

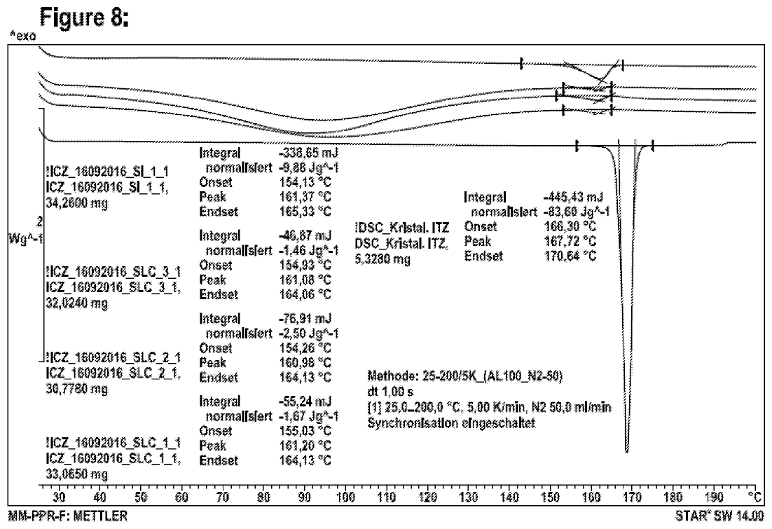

The dissolution of the nano-milled drug without stabilizer applied to a carrier as Parteck® SLC 500 and Kieselgel SI 5000 showed a very similar release property with or without stabilizer (FIG. 7). In both cases the nano-milled drug loaded carrier showed faster drug release as the pure crystalline drug.

In summary it is found that nano-milling without the addition of any stabilizers is possible, however, the active substance floats on top and no further workable nanosuspension is obtained. By adding a small amount of HPMC, “floating” can be prevented and the production of a nanosuspension is possible. In all cases the release of samples of the nano-milled drug loaded on the carriers was faster (in approx. 10 minutes) in comparison with the pure crystalline drug samples that do not reaches the max. solubility possible even after the 2 hours tested.

Comparison with Nanosuspensions with Itraconazole (Representative Example for Weak Bases) as Active Ingredient

In the same way as in the previous experiments, an itraconazole nanosuspension is prepared here, which is applied to Parteck® SLC 500. It is to be investigated if an improvement of the release can be achieved by the application of a nanosuspension.

The results obtained are compared with the results of the fenofibrate nanosuspension.

In comparison, Kieselgel SI 5000 is loaded with the crystalline itraconazole nanosuspension. Here, the influence of the pore diameter on the release of the itraconazole nanosuspension will be investigated. In addition, goal is to verify analytical results of itraconazole loaded carrier achieved, with the fenofibrate loaded carrier, to confirm conclusion that release of API nano-milled loaded carrier of different API is faster as crystalline API without milling and is independent from pore-diameter.

Procedure

At the beginning of the experiments, an itraconazole suspension is prepared, which is stabilized with hydroxypropylmethyl cellulose (HPMC) and DOSS and then nano-milled. The nanosuspension obtained is stored in the refrigerator between 2 and 8° C.

Using the impregnation method, the carriers Parteck® SLC 500 and Kieselgel SI 5000 are loaded in a ratio of 1:1 (w/w) with the itraconazole nanosuspension. The drying takes place in freeze-drying (see program “Nanosus_PK”). As drying in the program “Nanosus_PK” is not sufficient, it is dried again (program: Nanosus_PK_modified). Due to the hygroscopic properties of the Parteck® SLC 500, all batches produced are stored in the desiccator over orange gel.

TABLE 12
Batch overview

| batch | carrier | Drug (API) |
|---|---|---|
| ICZ_16092016_SLC_1 | Parteck ® SLC 500 | Itraconazole |
| ICZ_16092016_SLC_2 | Parteck ® SLC 500 | Itraconazole |
| ICZ_16092016_SLC_3 | Parteck ® SLC 500 | Itraconazole |
| ICZ_16092016_SI_1 | Kieselgel SI 5000 | Itraconazole |

Performed Measurements

All batches

Loss on drying
Differential Scanning Calorimetry
Release
Determination of content by HPLC
Determination of content by H₁-NMR

The measurements and determinations are carried out in the same way and with the same equipment and means as previously described.

TABLE 13
Used Materials

| Product | Origin |
|---|---|
| Itraconazole | Metrochem API Private limited |
| Hypromellose (HPMC) | Shin Etsu Chemical Co., Ltd. |
| (Pharmacoat 603) | |
| Dioctylsulfosuccinate | Aldrich Chemistry |
| sodium = DOSS | |
| Parteck ® SLC 500 | Merck KGaA |
| Kieselgel SI 5000 | Merck KGaA (Synthesis) |
| Milli-Q Water | Merck KGaA |

To stabilize the nanosuspension, hydroxypropylmethyl cellulose (HPMC) and DOSS are added to the suspension medium. It could be expected that without DOSS as stabilizing agent, the produced itraconazole nanosuspension would likely rapidly tend to aggregate and build up larger particles due to greatly increased surface effects, such as electrostatic attraction and dissolution rate (Ostwald ripening).

1.)

Preparation of Itraconazole Suspensions:
Itraconazole 19.870 g
HPMC 2.496 g
DOSS 0.500 g
Milli-Q water 77.020 g
Equipment: magnetic stirrer (IKA®-Werke GmbH & CO. KG, Staufen, Germany)
Rotation speed: 600 rpm
Temperature: off
Others: stirring fish, beaker, spatula

HPMC and DOSS are weighed into VWR screw-cap glass (250 mL) and supplemented with Milli-Q water to 80.016 g (weighed in, see above) to prepare the itraconazole suspension for nanomilling. The mixture is stirred with the magnetic stirrer and with the stirring fish for about 2 hours until completely dissolving. The screw jar is then closed.

The next day, shortly before milling, itraconazole is weighed. With stirring, the itraconazole is added and suspended for about 10 minutes. Subsequently, the suspension is milled in the nanomill.
Theoretical Itraconazole content: 19.89% (w/w)

2.)

Prepare Saturated Itraconazole Suspension in SGFsp:
Itraconazole about 200 mg
SGFsp 100 mL
Equipment: magnetic stirrer (IKA®-Werke GmbH & CO. KG, Staufen, Germany)
Rotation speed: 450 rpm
Temperature: 40° C. (thermostat)
Others: stirring fish, beaker, tablespoon
Time: 72 h

Approximately 200 mg of itraconazole are added to 100 mL SGFsp and suspended at 40° C. at 450 rpm for 72 h. The beaker is screwed during this time. Subsequently, the saturation concentration is measured by HPLC.

Nano-Milling
Equipment: Dyno®-Mill Research Lab (Willy A. Bachofen AG—Maschinenfabrik, Muttenz, Switzerland)
Weighed amount: 100 g
Time: 30 min
Milling balls: 55.0 mL zirconium oxide balls (SiLi ZYP 0.2-0.3 mm, Sigmund Lindner GmbH, Warmensteinach, Germany)
Temperature: −2° C. (cryostat)
Rotation speed: 3000 Upm
Taking samples: t=0, 10, 20, 30 min

To prepare the nanosuspension, the suspension to be placed in the hopper of the nanomill and the milling process is started. The temperature of the cryostat should be around 2° C. during milling. At the defined times, a few drops of the suspension are removed from the feed hopper using a disposable pipette and the particle size is measured by means as described above. When the desired particle size is reached, the grinding process is finished. The measurement of the particle size takes place at regular intervals and the particle size determination is carried out according to the methods described above.

Loading of Parteck® SLC 500/Kieselgel SI 5000 with Liaconazole and Production of Following Batches

TABLE 14
The loading is carried out as described above and the
batches are produced using the following amounts:

| | Silica | Suspension |
|---|---|---|
| batch | [g] | [g] |
| ICZ_16092016_SLC_1 | 10.00 | 10.00 |
| ICZ_16092016_SLC_2 | 10.06 | 9.84 |
| ICZ_16092016_SLC_3 | 10.00 | 9.93 |
| ICZ_16092016_SI_1 | 9.99 | 9.87 |

Freeze-Drying of Resulting Loaded Carriers
Device: Freeze Dryer Gamma 2-16 LSC (Christ Gefriertrocknungsanlagen Martin Christ, Osterode, Germany)
Cooling: water cooling

The loaded, moist products from table 14 are freeze-dried under the following conditions in a beaker:

TABLE 15
a) Program 1: “Nanosus PK”

| | |
|---|---|
| Temperature | Time |
| [° C.] | [h] |

| | | | |
|---|---|---|---|
| Freezing | −45° | C. | 8 |
| First drying | −35° | C. | 30 |
| Second drying | 0° | C. | 4 |
| | +25° | C. | 18 |

| | |
|---|---|
| Total | 60 |

TABLE 16
b) Program 3: “Nanosus PK modified”

| | |
|---|---|
| Temperature | Time |
| [° C.] | [h] |

| | | | |
|---|---|---|---|
| Freezing | −45° | C. | 5 |
| Main drying | −35° | C. | 30 |
| Second drying | 0° | C. | 4 |
| | +25° | C. | 18 |

| | |
|---|---|
| Total | 57 |

The samples are placed into the freeze dryer for freeze drying and the freeze dryer is closed.

Water is used for cooling (first the drain is turned on, only then the inlet!). Then the desired program is started. After freeze-drying, the drying loss of the product should be determined as described. If drying is insufficient, further drying is carried out.

Since the drying loss of all batches after drying with program 1 is about −10%, they are dried again. Program 3: Nanosus_PK_modified is used for this purpose. After repeating the drying, the drying loss is again measured and was below 3%. The drying loss of the Kieselgel SI 5000 is thereafter only −2%.

The drying loss is determined as described above. The drying loss should ideally be below 3% for release. If the drying loss is higher, a further drying may be necessary.

Differential Scanning Calorimetry is Carried Out as Described Before, but According to Program 1:
Methods: Program 1: (“25-200/5K_(Alu100_N-2)”)
Continuous heating of the sample from 25° C. to 200° C. at a heating rate of 5 K/min

Release of active ingredient is determined using the same device as applied before (Sotax 1 and 2; Freisetzungsapparatur Sotax AT 7smart (Sotax AG, Lörrach, Germany)

SGFsp is used as the medium and the release determinations are carried out at a wavelength of 225 nm. Each sample is collected in a test tube with the automatic sampler. Subsequently, the content of the samples is determined offline by HPLC. Advantageously the nano-milled loaded samples do not float on the release medium like the crystalline active substance but are better wetted.

Determination of Content by HPLC
Device: HPLC-system LaChrom Elite (Hitachi Europe GmbH, Düsseldorf, Germany)
Detector: UV Detector L-2400 VWR Hitachi (Hitachi Europe GmbH, Düsseldorf, Germany)
Autosampler: Autosampler L-2200 VWR Hitachi (Hitachi Europe GmbH, Düsseldorf, Germany)
Column: Chromolith Performance RP-18e 100-4.6 mm (OB1108048)
Eluent: Acetonitrile/TBAHS-Buffer (1.7 g/L)/Methanol (450:450:100)
Wash solution—sampler: Acetonitrile/Milli-Q-Wasser (1:1)
Column oven—temperature: 25° C.
Injection volume: 15 μL
Wave length—detector: 225 nm
Flow rate: 2.0 mL/min (isocratic)
Duration—run: 7 min
Sequence: XXXXXXXX_01_PK_Itaconazole_Disso_Nanosuspensionen 1. Seq
Method: XXXXXXXX_01_PK_Itaconazole_Method
Filter—sample preparation: Whatman™ Anotop™ 10, 0.02 μm, Cat.-No.: 6809-1002

Prior to filling into vials, each sample from the release is first filtered with a syringe with Luer-Lock connection and above filters for sample preparation to retain any particles of the nanosuspension and eliminate a systematic error.

For evaluation of the HPLC results, the saturation concentration is determined and the release of crystalline Itraconazole from the experiment is taken.

H₁-NMR:
Method: 1H-NMR spectroscopy
Conditions: DMSO-d6
Measurement modus: content [%]

Results:

Particle Size Distribution of the Nanosuspension

The particle size distribution is measured with a Zetasizer Nano SZ.

The particle size is measured immediately after the preparation of the nanosuspension.

The particle size remains almost constant over a longer period, the crystals in suspension grow only very slowly. This means that the nanosuspension is sufficiently well stabilized by the use of HPMC and DOSS.

Optical Assessment of the Batches Produced

ICZ_16092016_SLC_1

After loading the Parteck® SLC 500 with the itraconazole nanosuspension, the support material is slightly clumped. After freeze-drying, these lumps remain. They are easy to be divided with the spatula. The remainder, loose powder is of fine consistency. The color is white as that of the starting substance.

ICZ_16092016_SLC_2

The loading of the Parteck® SLC 500 also leads to the formation of smaller lumps in the second batch. As with the first batch, the lumps can be easily crushed with the spatula.

ICZ_16092016_SLC_3

Just like the other batches, the Parteck® SLC 500 also has some lumps that can be easily crushed with the spatula after impregnation.

ICZ_16092016_SI_1

Similar as the loading of the Parteck® SLC 500, the loading of the Kieselgel SI 5000 produces some lumps of different sizes. Compared to Parteck® SLC 500, the loose, remaining powder is floury-like.

Determination of Content by H₁-NMR

TABLE 17
By the external H₁-NMR content determination of the samples from the
different batches shows the following active ingredient contents:

| | Theoretical | Measured |
|---|---|---|
| | content | content |
| batch | [%] | [%] |
| ICZ_16092016_SLC_1 | 16.08 | 13.3 |
| ICZ_16092016_SLC_2 | 16.09 | 13.2 |
| ICZ_16092016_SLC_3 | 16.20 | 12.6 |
| ICZ_16092016_SI_1 | 16.21 | 15.2 |

DSC Measurement

TABLE 18
physical status amorphous/crystalline

| batch | excipient | crystallinity |
|---|---|---|
| ICZ_16092016_SLC_1 | Parteck ® SLC 500 | crystalline |
| ICZ_16092016_SLC_2 | Parteck ® SLC 500 | crystalline |
| ICZ_16092016_SLC_3 | Parteck ® SLC 500 | crystalline |
| ICZ_16092016_SI_1 | Kieselgel SI 5000 | crystalline |

This FIG. 8 shows the DSC curve of crystalline itraconazole, along with the curves of the nanosuspension-loaded batches of Parteck® SLC 500 and Kieselgel SI 5000. The endothermic melting peak of the pure itraconazole, starting at approx. 166° C., is clearly visible. The melt peaks of the nanosuspension batches on Parteck® SLC 500 and Kieselgel SI 5000 are much less pronounced and is shifted to lower temperatures. Instead of a sharp melting peak of itraconazole a broadened melting peak occurs for the loaded batches which begins earlier, this means at about 155° C.

Since the nanosuspension is not just pure itraconazole, both the hydroxypropylmethyl cellulose used and DOSS can cause a melting point depression.

In addition, a “masking” of the heat transfer could take place through the silica supports used, so that a defined, clear melting peak is covered. The active ingredient is found in crystalline form in all samples.

Release of Active Ingredient Itraconazole Loaded on Different Carrier

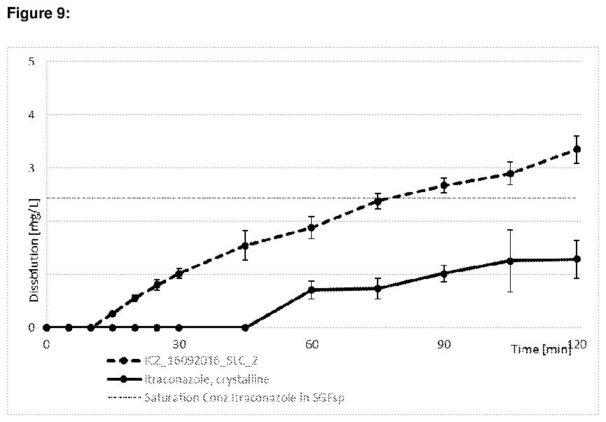

FIG. 9 shows the results of the batch, ICZ_16092016_SLC_2, which releases with a maximum concentration of approx. 3 mg/L and substantial faster than itraconazole crystalline sample compared. The saturation solubility is exceeded by about 0.5 mg/L due to challenges in analytical evaluation.

While crystalline itraconazole floats on the release medium, all samples of loaded Parteck® SLC 500 batches drop rapidly to the bottom of the vessel after adding. Only a small part of the sample floats on the surface of the medium.

Analytical results of itraconazole nano-milled loaded on different carrier supported evaluation of fenofibrate nano-milled loaded carrier reported before. In all case API (representative of API natures) loaded on different carrier results in substantial faster release in comparison to the pure crystalline API.

1. A method for producing a pharmaceutical composition characterized by the following steps
a) an active ingredient is brought into suspension in a solvent or a solvent mixture,
b) the prepared suspension is milled at a temperature below 0° C. to a mean particle diameter of the active ingredient of less than 200 nm,
c) the resulting suspension is mixed with a carrier material and
d) the solvent is removed and the active ingredient is adsorbed on the carrier.

2. Method according to claim 1, characterized in that the active ingredient is a poorly soluble and/or low bioavailable ingredient of substance classes BCS class II or IV.

3. Method according to claim 1, characterized in that the active ingredient is selected from the group of acidic or basic agents.

4. Method according to claim 1, characterized in that in step a) the active ingredient is suspended in water.

5. Method according to claim 1, characterized in that in step a) the suspension is stabilized by the addition of at least one stabilizer selected from the group Hydroxypropylmethylcellulose (HPMC) and sodium dioctylsulfosuccinate (DOSS).

6. Method according to claim 1, characterized in that in step d) the solvent is removed by freeze drying.

7. Method according to claim 1, characterized in that in step c) the suspension is mixed with a silica gel as carrier material.

8. Method according to claim 1, characterized in that in step c) the suspension is mixed with a silica gel as carrier, having a specific surface area in the range of about 1 m²/g to about 600 m²/g (BET measurement) and an average pore size of about 2 to 600 nm.

9. Method according to claim 1, further comprising granulation, capsule filling or tableting.

10. Pharmaceutical formulation containing a pharmaceutical composition obtainable by a method according to claim 1.

11. Pharmaceutical formulation according to claim 10, wherein the pharmaceutical composition has an improved release profile.

12. Pharmaceutical formulation of claim 10, formulated in powders, capsules, granules, coated granules, tablets or coated tablets.

13. The method according to claim 1, wherein in b) the prepared suspension is milled at a temperature below 0° C. to a mean particle diameter of the active ingredient in a range from 60 to 160 nm,

14. The method according to claim 1, wherein in step c) the suspension is mixed with a silica gel as carrier, having a specific surface area in the range of about 3 m²/g to about 500 m²/g (BET measurement) and an average pore size of about 6 to 500 nm.
